(19) United States
(12) Patent Application Publication
(10) Pub. No.: US 2023/0239259 A1
(43) Pub. Date: Jul. 27, 2023

(54) MESSAGE SELECTION APPARATUS, MESSAGE PRESENTATION APPARATUS, MESSAGE SELECTION METHOD, AND MESSAGE SELECTION PROGRAM

(71) Applicant: NIPPON TELEGRAPH AND TELEPHONE CORPORATION, Tokyo (JP)

(72) Inventors: Mana SASAGAWA, Musashino-shi, Tokyo (JP); Tae SATO, Musashino-shi, Tokyo (JP)

(73) Assignee: NIPPON TELEGRAPH AND TELEPHONE CORPORATION, Tokyo (JP)

(21) Appl. No.: 18/008,580

(22) PCT Filed: Jun. 8, 2020
(86) PCT No.: PCT/JP2020/022489

§ 371 Date: Dec. 6, 2022

(51) Int. Cl. H04L 51/04; A61B 5/16; G06F 40/30

(52) U.S. Cl. CPC H04L 51/04; A61B 5/165; G06F 40/30

(57) ABSTRACT

A message selection device according to an embodiment includes a message database that holds a plurality of messages in correspondence with each of various emotion of a communicator, a communicator information acquisition unit configured to acquire communicator information for estimating the emotion of the communicator, a receiver information acquisition unit that acquires receiver information for estimating the emotion of a receiver who is to receive a message from the communicator, and a message selection unit that estimates the emotion of the communicator based on the communicator information acquired by the communicator information acquisition unit, estimates the emotion of the receiver based on the receiver information acquired by the receiver information acquisition unit, and selects a message from among the messages held by the message database based on the estimated emotions.

### TECHNICAL FIELD

Embodiments of the present invention relate to a message selection device, a message presentation device, a message selection method, and a message selection program.

### BACKGROUND ART

Various techniques for presenting a message based on a communicator's emotion have been proposed.

For example, PTL 1 discloses an emotion estimation technique for estimating a dog's emotion based on characteristics of the dog's bark. Products that apply this emotion estimation technique to provide tools for communication with pets are also available. With such a product, multiple messages are prepared for each of various pet emotions, and a message associated with an estimated emotion is randomly presented.

### CITATION LIST

### Patent Literature

[PTL 1] WO 2003/015076

### SUMMARY OF THE INVENTION

It is said to be important in communication for both the communicator and the receiver to take each other's emotions into consideration and, in some cases, sympathize with the emotions of the other party.

PTL 1 does not disclose a configuration with consideration given to the emotions of the receiver who is the communication partner.

The present invention is directed to providing technology that makes it possible to select a message for presentation with consideration given to not only the emotion of the communicator but also the emotion of the receiver who is the communication partner.

### Means for Solving the Problem

In order to solve the foregoing problems, a message selection device according to an aspect of the present invention includes: a message database configured to hold a plurality of messages in correspondence with an emotion of a communicator; a communicator information acquisition unit configured to acquire communicator information for estimating the emotion of the communicator; a receiver information acquisition unit configured to acquire receiver information for estimating an emotion of a receiver who is to receive a message from the communicator; and a message selection unit configured to estimate the emotion of the communicator based on the communicator information acquired by the communicator information acquisition unit, estimate the emotion of the receiver based on the receiver information acquired by the receiver information acquisition unit, and select a message from the plurality of messages held by the message database based on the estimated emotions.

### Effects of the Invention

According to this aspect of the present invention, it is possible to provide technology that makes it possible to select a message for presentation with consideration given to not only the emotion of the communicator but also the emotion of the receiver who is the communication partner.

### BRIEF DESCRIPTION OF DRAWINGS

FIG. 1A is a block diagram showing an example of the configuration of a message presentation device that includes a message selection device according to a first embodiment of the present invention.

FIG. 1B is a diagram showing an example of the hardware configuration of an information processing device that constitutes the message presentation device.

FIG. 2 is a diagram showing an example of information held in a message database.

FIG. 3 is a flowchart showing an example of processing operations in the information processing device.

FIG. 4A is a diagram in which emotion components indicated by the message “Wow! Great!” are modeled on Russell's circumplex model of affect.

FIG. 4B is a diagram in which emotion components indicated by the message “I'm kind of excited!” are modeled on Russell's circumplex model of affect.

FIG. 5A is a diagram showing an emotion vector of the message “Wow! Great!” acquired based on the emotion vectors shown in FIG. 4A.

FIG. 5B is a diagram showing an emotion vector of the message “I'm kind of excited!” acquired based on the emotion vectors shown in FIG. 4B.

FIG. 6A is a diagram showing the relationship between the emotion vector of the message shown in FIG. 5A and an emotion vector of a receiver emotion.

FIG. 6B is a diagram showing the relationship between the emotion vector of the message shown in FIG. 5B and an emotion vector of a receiver emotion.

FIG. 7 is a block diagram showing an example of the configuration of a message presentation device that includes a message selection device according to a second embodiment of the present invention.

FIG. 8 is a diagram showing an example of the hardware configuration of an information processing device that constitutes a communicator device in the message presentation device shown in FIG. 7.

FIG. 9A is a flowchart showing an example of processing operations in the information processing device shown in FIG. 8.

FIG. 9B is a flowchart showing an example of processing operations in an information processing device that constitutes a receiver device in the message presentation device shown in FIG. 7.

FIG. 10 is a block diagram showing an example of the configuration of a message presentation device that includes a message selection device according to a third embodiment of the present invention.

FIG. 11 is a block diagram showing the configuration of the receiver device in another example of the configuration of the message presentation device that includes the message selection device according to the third embodiment.

FIG. 12 is a diagram showing an example of information held in a ratio database in the message selection device according to the third embodiment.

### DESCRIPTION OF EMBODIMENTS

Hereinafter, embodiments of the present invention will be described with reference to the drawings.

### First Embodiment

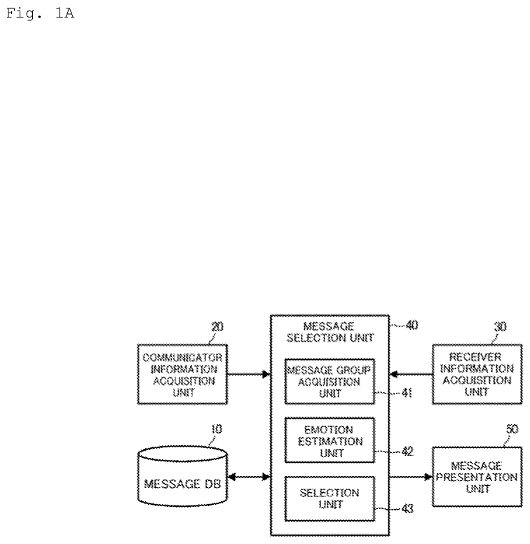

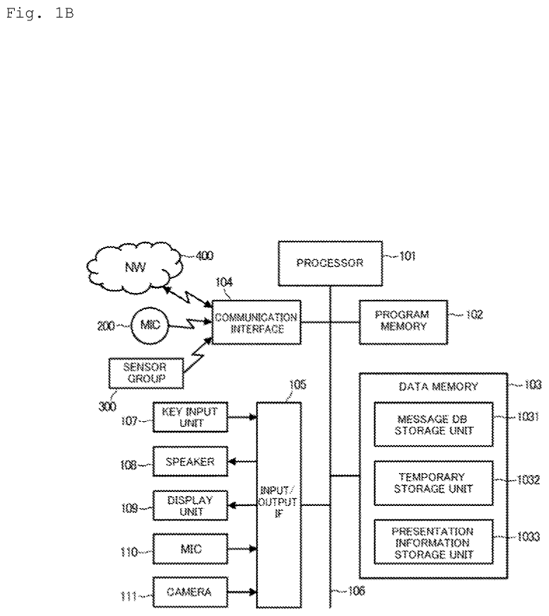

FIG. 1A is a block diagram showing an example of the configuration of a message selection device that includes a message generation device according to a first embodiment of the present invention. The message selection device includes a message database 10, a communicator information acquisition unit 20, a receiver information acquisition unit 30, and a message selection unit 40. The message presentation device includes a message selection device and a message presentation unit 50. Note that in FIG. 1A, “message database” is abbreviated as “message DB”.

Here, the message database 10 holds a plurality of messages corresponding to communicator emotions.

The communicator information acquisition unit 20 acquires communicator information for estimating an emotion of the communicator. Examples of the communicator include pets that make various calls and whines depending on their emotions, such as a dog, a cat, or a bird. The communicator may also be a human infant who is still unable to speak and expresses their emotions by crying or whining. The communicator information includes at least vocalization information about a vocalization emitted by the communicator. The communicator information can also include various types of information that can be used to estimate the emotion of the communicator, such as image information that captures the appearance of the communicator, and biometric information that indicates a biological state such as the communicator's body temperature and heart rate.

The receiver information acquisition unit 30 acquires receiver information for estimating the emotion of the receiver who receives the message from the communicator. Examples of the receiver include pet owners and parents of human infants. The receiver information can include various types of information that can be used to estimate the emotion of the receiver, such as vocalization information regarding a vocalization made by the receiver, image information that captures the appearance of the receiver, and receiver biometric information.

The message selection unit 40 estimates the emotion of the communicator based on the communicator information acquired by the communicator information acquisition unit 20, estimates the emotion of the receiver based on the receiver information acquired by the receiver information acquisition unit 30, and selects one message from a plurality of messages held in the message database 10 based on the estimated emotions.

More specifically, the message selection unit 40 includes a message group acquisition unit 41, an emotion estimation unit 42, and a selection unit 43.

The message group acquisition unit 41 acquires a message group that corresponds to the communicator information acquired by the communicator information acquisition unit 20 from the message database 10.

The emotion estimation unit 42 estimates emotions indicated by the messages in the message group acquired by the message group acquisition unit 41, and also estimates an emotion indicated by the receiver information acquired by the receiver information acquisition unit 30.

Based on the emotions indicated by the messages in the message group that were estimated by the emotion estimation unit 42, the selection unit 43 selects the message that is closest to the receiver emotion estimated by the emotion estimation unit 42.

Also, the message presentation unit 50 presents the message selected by the message selection unit 40 to the receiver.

FIG. 1B is a diagram showing an example of the hardware configuration of an information processing device that constitutes the message presentation device shown in FIG. 1A. The information processing device may be provided as a communication device that includes a dedicated housing, or may be realized by a general-purpose computer such as a smartphone or a personal computer.

As shown in FIG. 1B, the information processing device includes a hardware processor 101 such as a CPU (Central Processing Unit). In the information processing device, a program memory 102, a data memory 103, a communication interface 104, and an input/output interface 105 are connected to the processor 101 via a bus 106. Note that in FIG. 1B, “input/output interface” is abbreviated as “input/output IF”.

Here, the program memory 102 is a non-transitory tangible computer-readable storage medium that includes a non-volatile memory that can be written to and read from at any time, such as an HDD (Hard Disk Drive) or an SSD (Solid State Drive), in combination with a non-volatile memory such as a ROM (Read Only Memory). The program memory 102 stores programs necessary for the processor 101 to execute various types of control processing pertaining to the first embodiment. Specifically, processing function units in the communicator information acquisition unit 20, the receiver information acquisition unit 30, the message selection unit 40, and the message presentation unit 50 can all be realized by the processor 101 reading out and executing a program stored in the program memory 102. Note that some or all of these processing function units may be realized in various other aspects, including an integrated circuit such as an application specific integrated circuit (ASIC), a DSP (Digital Signal Processor), or an FPGA (Field-Programmable Gate Array).

Also, the data memory 103 is a tangible computer-readable storage medium that includes the above-mentioned non-volatile memory in combination with a volatile memory such as a RAM (Random Access Memory). The data memory 103 is used to store various types of data acquired and created during the execution of various types of processing. Specifically, areas for storing various types of data are appropriately secured in the data memory 103 during the execution of various types of processing. As examples of such areas, the data memory 103 may be provided with a message database storage unit 1031, a temporary storage unit 1032, and a presentation information storage unit 1033. Note that in FIG. 1B, “message database storage unit” is abbreviated as “message DB storage unit”.

The message database storage unit 1031 stores a plurality of messages in correspondence with each of various communicator emotions. Specifically, the message database 10 can be configured in the message database storage unit 1031.

The temporary storage unit 1032 stores various types of data, such as data that is acquired or generated when the processor 101 performs operations as the communicator information acquisition unit 20, the receiver information acquisition unit 30, and the message selection unit 40, as well as communicator information, receiver information, message groups, and emotions.

The presentation information storage unit 1033 stores a message that is selected when the processor 101 performs operations as the message selection unit 40 and that is to be presented to the receiver when the processor 101 performs operations as the message presentation unit 50.

The communication interface 104 can include one or more wired or wireless communication modules.

As one example, the communication interface 104 includes a wireless communication module that utilizes short-range wireless technology such as Bluetooth (registered trademark). This wireless communication module receives vocalization signals from a wireless microphone 200, sensor signals from sensors in a sensor group 300, and the like Under control of the processor 101. Note that in FIG. 1B, “wireless microphone” is abbreviated as “MIC”. The wireless communication module can convert the received signals into information that can be processed by the processor 101, and can store such information in the temporary storage unit 1032 of the data memory 103. For example, if the wireless microphone 200 is attached to the communicator or arranged in the vicinity of the communicator, the information processing device can acquire vocalization information regarding the communicator. Also, if an image sensor such as a camera included in the sensor group 300 is arranged so as to capture images of the communicator, or if a biometric sensor such as a body temperature sensor or a heart rate sensor is attached to the communicator, the information processing device can acquire image information or biometric information regarding the communicator. In other words, the processor 101 and the communication interface 104 can function as the communicator information acquisition unit 20. Also, if a biometric sensor such as a body temperature sensor or a heartbeat sensor included in the sensor group 300 is attached to the receiver, the information processing device can acquire biometric information regarding the receiver. In other words, the processor 101 and the communication interface 104 can also function as the receiver information acquisition unit 30.

Also, the communication interface 104 may include a wireless communication module that wirelessly connects to a Wi-Fi access point or a mobile phone base station, for example. Under control of processor 101, the wireless communication module can perform communication with other information processing devices and server devices on the network 400 via Wi-Fi access points or mobile phone base stations, and transmit and receive various types of information. Note that in FIG. 1B, “network” is abbreviated as “NW”. The network 400 is constituted by an IP network including the Internet, and an access network for accessing the IP network. Examples of the access network include a public wired network, a mobile phone network, a wired LAN (Local Area Network), a wireless LAN, and a CATV (Cable Television) network.

Also, a key input unit 107, a speaker 108, a display unit 109, a microphone 110, and a camera 111 are connected to the input/output interface 105. Note that in FIG. 1B, “microphone” is abbreviated as “MIC”.

The key input unit 107 includes operation keys and buttons for allowing the receiver, who is a user of the information processing device, to give operation instructions to the processor 101. In response to operations performed on the key input unit 107, the input/output interface 105 inputs corresponding operation signals to the processor 101.

The speaker 108 generates sound in accordance with a signal received from the input/output interface 105. For example, the processor 101 converts a message stored in the presentation information storage unit 1033 into vocalization information, and the vocalization information is input to the speaker 108 as an audio signal by the input/output interface 105, and thus the message is presented to the receiver as audio. In other words, the processor 101, the input/output interface 105, and the speaker 108 can function as the message presentation unit 50.

The display unit 109 is a display device that uses a liquid crystal display, an organic EL (Electro Luminescence) display, or the like and displays images that correspond to signals received from the input/output interface 105. For example, the processor 101 converts the message stored in the presentation information storage unit 1033 into image information, and the image information is input to the display unit 109 as an image signal by the input/output interface 105, and thus the message can be presented to the receiver as an image. In other words, the processor 101, the input/output interface 105, and the display unit 109 can function as the message presentation unit 50. Note that the key input unit 107 and the display unit 109 may be configured as an integrated device. Specifically, it may be a so-called tablet input/display device in which an electrostatic-capacitance or pressure-sensitive input detection sheet is arranged on the display screen of a display device.

The microphone 110 collects nearby sounds and inputs them as an audio signal to the input/output interface 105. Under control of the processor 101, the input/output interface 105 converts the received audio signal into vocalization information and stores it in the temporary storage unit 1032. If the information processing device is located near the receiver such as in the case of being a smartphone, the microphone 110 collects vocalizations emitted by the receiver. Therefore, the processor 101 and the input/output interface 105 can function as the receiver information acquisition unit 30. Also, if the distance between the receiver and the communicator is short and the microphone 110 can collect vocalizations from both the receiver and the communicator, the processor 101 and the input/output interface 105 can function as the communicator information acquisition unit 20. For example, using a feature quantity of the frequency or the like of the vocalization information, the processor 101 can handle the vocalization information as sentence information and perform speech recognition to obtain the meaning to some extent, and under some conditions can determine whether the vocalization information is receiver information or communicator information.

The camera 111 captures images in the field of view and inputs a captured image signal to the input/output interface 105. Under control of the processor 101, the input/output interface 105 converts the received captured image signal into image information and stores the image information in the temporary storage unit 1032. If the receiver is in the field of view of the camera 111, the processor 101 and the input/output interface 105 can function as the receiver information acquisition unit 30 that acquires receiver image information. Also, if the communicator is in the field of view of the camera 111, the processor 101 and the input/output interface 105 can function as the communicator information acquisition unit 20 that acquires communicator image information. The processor 101 can determine whether image information is receiver information or the communicator information based on a feature quantity of the image information, for example.

The input/output interface 105 may have a function for reading from and writing to a recording medium such as a semiconductor memory (e.g., a flash memory), or may have a function for connection to a reader/writer having a function for reading from and writing to such a recording medium. Accordingly, a recording medium that can be mounted to and removed from the information processing device can be used as the message database storage unit that stores messages. The input/output interface 105 may further have a function for connection with other devices.

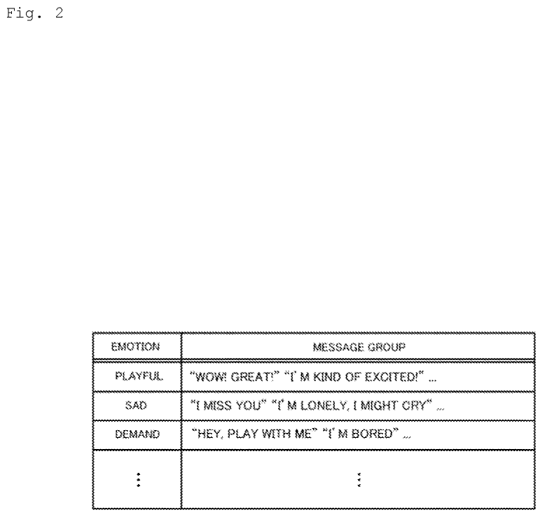

FIG. 2 is a diagram showing an example of information held in the message database 10 configured in the message database storage unit 1031. This is an example in the case where the communicator is a dog and the receiver is a human. As shown in FIG. 2, the message database 10 stores a message group containing a plurality of messages in correspondence with each of various emotions of the dog (communicator) such as “playful”, “sad”, and “demand”. For example, a message group including messages such as “Wow! Great!” and “I'm kind of excited!” is stored in correspondence with the emotion “playful”.

Next, operations of the message presentation device that includes the message selection device will be described. The case where the communicator is a dog and the receiver is a human is described in the following example.

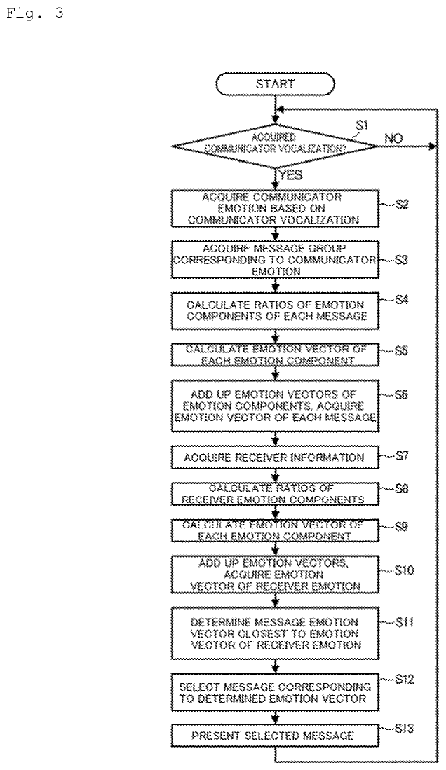

FIG. 3 is a flowchart showing an example of processing operations in the message presentation device. This flowchart shows processing operations in the processor 101 of the information processing device that functions as the communicator information acquisition unit 20, the receiver information acquisition unit 30, the message selection unit 40, and the message presentation unit 50 of the message presentation device. For example, if the wireless microphone 200 is attached to the dog, which is the communicator, or in the vicinity thereof and then an instruction for starting message presentation is received from the key input unit 107 via the input/output interface 105, the processor 101 starts to perform the operations shown in the aforementioned flowchart. Note that the sensor group 300 that includes an image sensor, a biometric sensor, and the like may be used in addition to the wireless microphone 200, but in this example, the dog's emotion is estimated using only the dog's bark as communicator information.

First, the processor 101 functions as the communicator information acquisition unit 20 and determines whether or not a communicator vocalization collected by the wireless microphone 200, that is to say dog's bark, has been acquired by the communication interface 104 (step S1). Here, if it is determined that a communicator vocalization has not been acquired (NO in step S1), the processor 101 repeats the processing of step S1.

On the other hand, if it is determined that a communicator vocalization has been acquired (YES in step S1), the processor 101 stores the acquired communicator vocalization in the temporary storage unit 1032 and performs operations as the message group acquisition unit 41 of the message selection unit 40.

Specifically, first, the processor 101 acquires a communicator emotion, that is to say the dog's emotion, based on the communicator vocalization stored in the temporary storage unit 1032 (step S2). There are no particular limitations on the method used to acquire the communicator emotion in this embodiment. For example, the emotion of the dog can be obtained using the method as disclosed in PTL 1.

The processor 101 then acquires a message group that corresponds to the acquired communicator emotion from the message database 10 stored in the message database storage unit 1031 and stores the acquired message group in the temporary storage unit 1032 (step S3).

Subsequently, the processor 101 performs operations as the emotion estimation unit 42.

Specifically, first, for each of the messages included in the message group stored in the temporary storage unit 1032, the processor 101 calculates the ratios of the emotion components indicated by the message (step S4). There are no particular limitations on the method for calculating the ratios of emotion components in this embodiment. For example, the ratios of emotion components can be calculated by an emotion component ratio calculating algorithm stored in the program memory 102 or the data memory 103. Text emotion recognition AI (e.g., https://emotion-ai.userlocal.jp/) is also available on the Internet as existing technology. In the case of using an emotion recognition resource provided on some sort of site on the Internet for calculating the ratios of emotion components in text, the processor 101 transmits, via the communication interface 104, the text of the message to a specified site on the network 400 that provides that resource. Accordingly, the processor 101 can receive emotion component ratio data corresponding to the transmitted text from the specified site.

For example, in the case of the message “Wow! Great!” in the message group that corresponds to the emotion “playful” shown in FIG. 2, the processor 101 calculates joy=0.83, favor=0.87, fear=0.05, sadness=0.04, and anger=0.55 as the ratios of emotion components. Also, in the case of the message “I'm kind of excited!”, the processor 101 calculates joy=0.66, favor=0.88, fear=0.04, sadness=0.09, and anger=0.07 as the ratios of the emotion components.

Next, the processor 101 converts the calculated emotion components into an emotion vector for each message (step S5). The emotion vector is a vector on Russell's circumplex model of affect. Russell's circumplex model of affect is a model that maps emotions in a two-dimensional space centered on valence and arousal. Russell's circumplex model of affect is disclosed in “J. A. Russell, ‘A circumplex model of affect’, Journal of Personality and Social Psychology, vol. 39, no. 6, p. 1161, 1980”, for example.

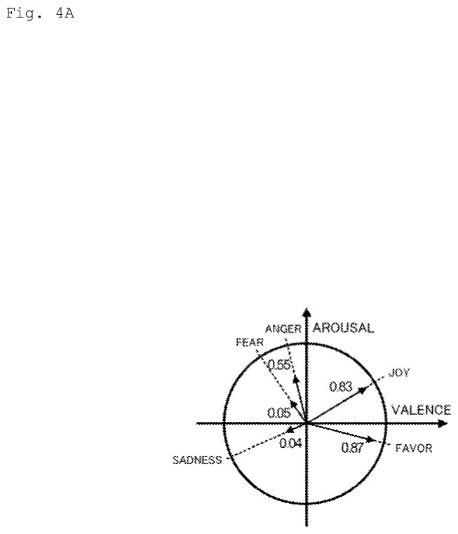

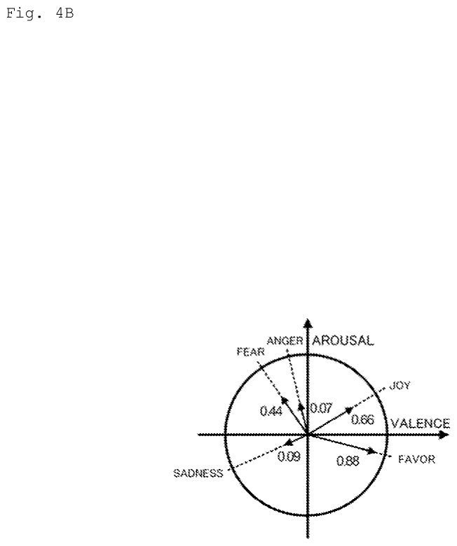

FIG. 4A is a diagram in which emotion components indicated by the message “Wow! Great!” are modeled on Russell's circumplex model of affect, and FIG. 4B is a diagram in which emotion components indicated by the message “I'm kind of excited!” are modeled on Russell's circumplex model of affect. In Russell's circumplex model of affect, on the valence axis, the degree of “pleasure” increases rightward and the degree of “misery” increases leftward. On the arousal axis, the degree of “arousal” increases upward and the degree of “sleepiness” increases downward. Each component of emotion is represented as a direction away from the origin in Russell's circumplex model of affect. For each vector in Russell's circumplex model of affect calculated by the processor 101, the magnitude of the vector (min0 to MAX1) is the calculated emotion ratio, and the direction of the vector is the direction, from the origin, to which the emotion was mapped.

The processor 101 then acquires an emotion vector for each message by obtaining the sum of the emotion vectors of the emotion components of the message (step S6). Concepts regarding emotion vectors and resultant force on Russell's circumplex model of affect are disclosed in “Reiko Ariga, Junji Watanabe, Junji Nunobiki, ‘Impression evaluation of emotional expressions of agents in response to expansion and contraction of graphic’, Human Interface Symposium 2017, Proceedings (2017)”, for example.

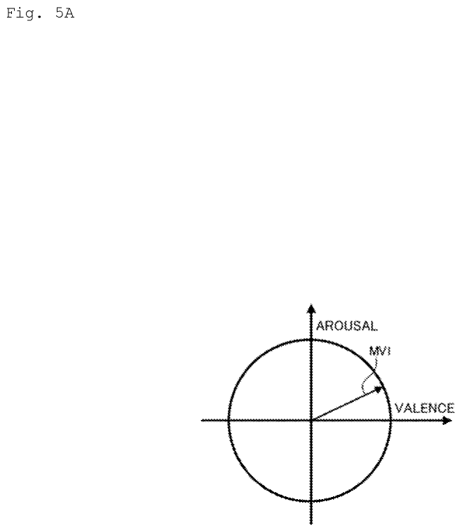

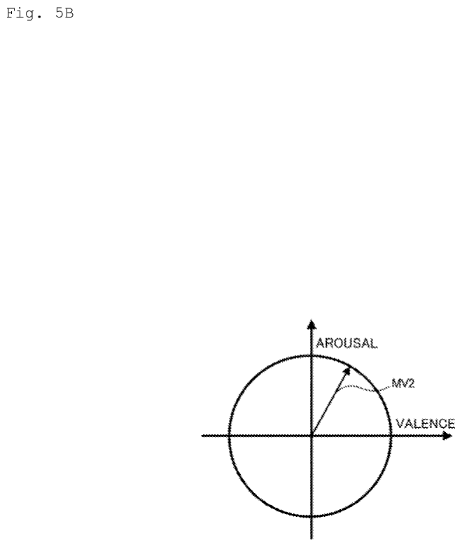

FIG. 5A is a diagram showing an emotion vector MV1 of the message “Wow! Great!” acquired based on the emotion vectors shown in FIG. 4A. Also, FIG. 5B is a diagram showing an emotion vector MV2 of the message “I'm kind of excited!” acquired based on the emotion vectors shown in FIG. 4B.

The processor 101 also acquires an emotion vector for the emotion of the human being who is the receiver.

To achieve this, the processor 101 first performs operations as the receiver information acquisition unit 30 and acquires receiver information (step S7). For example, a receiver vocalization collected by the microphone 110 and/or a face image of the human receiver captured by the camera 111 is stored as the receiver information in the temporary storage unit 1032 by the processor 101 via the input/output interface 105.

The processor 101 then performs operations as the emotion estimation unit 42 again, and acquires an emotion vector of the receiver emotion.

Specifically, first, the processor 101 calculates the ratios of emotion components of the person who is the receiver based on the vocalization and/or the face image stored in the temporary storage unit 1032 (step S8). There are also no particular limitations on the method for calculating the ratios of emotion components of the receiver in this embodiment. For example, a technique for calculating the ratios of emotion component based on a vocalization or a face image is disclosed in “Panagiotis Tzirakis, George Trigeorgis, Mihalis A. Nicolaou, Bjorn W. Schuller, Stefanos Zafeiriou, ‘End-to-End Multimodal Emotion Recognition Using Deep Neural Networks’, IEEE Journal of Selected Topics in Signal Processing, vol. 11, no. 8, pp. 1301-1309, 2017”. The processor 101 can calculate the ratios of emotion components using an emotion component ratio calculation algorithm stored in the program memory 102 or the data memory 103. Facial expression emotion recognition AI (e.g., https://emotion-ai.userlocal.jp/face) is also available on the Internet as existing technology. In the case of using an emotion recognition resource provided on some sort of site on the Internet for calculating the ratios of emotion components based on an expression, the processor 101 transmits, via the communication interface 104, the face image to a specified site on the network 400 that provides that resource. Accordingly, the processor 101 can receive emotion component ratio data corresponding to the transmitted face image from the specified site.

Next, the processor 101 converts the calculated receiver emotion components into an emotion vector (step S9).

The processor 101 then acquires an emotion vector of the emotion of the human being who is the receiver by obtaining the sum of the emotion vectors of the emotion components (step S10). Hereinafter, the emotion of the human being who is the receiver will be referred to as the “receiver emotion”.

In this way, after the emotion vector of each message of the message group and the emotion vector of the receiver emotion have been acquired, the processor 101 performs operations as the selection unit 43.

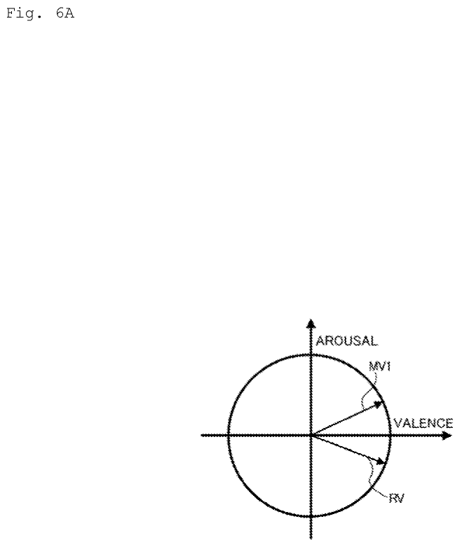

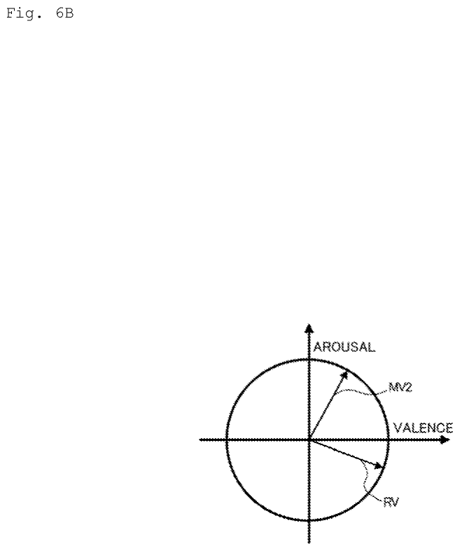

Specifically, first, the processor 101 determines the message emotion vector that is closest to the emotion vector of the receiver emotion (step S11). For example, the processor 101 determines the message emotion vector that has the largest inner product with the emotion vector of the receiver emotion. FIG. 6A is a diagram showing the relationship between the message emotion vector MV1 shown in FIG. 5A and the emotion vector RV of the receiver emotion, and FIG. 6B is a diagram showing the relationship between the message emotion vector MV2 shown in FIG. 5B and the emotion vector RV of the receiver emotion. In this example, the message emotion vector MV1 is closer to the emotion vector RV of the receiver emotion.

The processor 101 then selects the message that has the determined message emotion vector (step S12). In the above example, the processor 101 selects the message “Wow! Great!” that has the emotion vector MV1. The processor 101 stores the selected message in the presentation information storage unit 1033.

After a message has been selected from the message group in this way, the processor 101 functions as the message presentation unit 50 and presents the selected message (step S13). Specifically, the processor 101 presents the message stored in the presentation information storage unit 1033 by, via the input/output interface 105, outputting the message as audio with use of the speaker 108 or outputting the message as an image to the display unit 109.

Subsequently, the processor 101 repeats the processing from step S1.

The message selection device according to the first embodiment described above includes: a message database 10 configured to hold a plurality of messages in correspondence with an emotion of a communicator; a communicator information acquisition unit 20 configured to acquire communicator information for estimating the emotion of the communicator; a receiver information acquisition unit 30 configured to acquire receiver information for estimating an emotion of a receiver who is to receive a message from the communicator; and a message selection unit 40 configured to estimate the emotion of the communicator based on the communicator information acquired by the communicator information acquisition unit 20, estimate the emotion of the receiver based on the receiver information acquired by the receiver information acquisition unit 30, and select a message from the plurality of messages held by the message database 10 based on the estimated emotions. Accordingly, it is possible to select a message for presentation with consideration given to not only the emotion of the communicator but also the emotion of the receiver who is the communication partner.

Also, in the message selection device according to the first embodiment, the message selection unit 40 includes: a message group acquisition unit 41 configured to acquire, from the message database 10, a message group that corresponds to the communicator information acquired by the communicator information acquisition unit 20; an emotion estimation unit 42 configured to estimate an emotion indicated by each message in the message group acquired by the message group acquisition unit 41, and estimate an emotion indicated by the receiver information acquired by the receiver information acquisition unit 30; and a selection unit 43 configured to select a message that is closest to the emotion of the receiver estimated by the emotion estimation unit 42, based on the emotions indicated by the messages in the message group that were estimated by the emotion estimation unit 42. In this way, the message closest to the emotion of the receiver is selected from among the messages generated based on communicator emotions, and therefore it is possible to give consideration to not only the emotion of the communicator but also the emotion of the receiver and select a message that is closer to the receiver emotion, thus making it easier to provide a sense of sympathy with the receiver and also make communication smoother.

Also, in the message selection device according to the first embodiment, the emotion estimation unit 42 converts each message in the message group acquired by the message group acquisition unit 41 into an emotion vector, and converts the receiver information acquired by the receiver information acquisition unit 30 into an emotion vector, and the selection unit 43 determines, from among the emotion vectors of the messages in the message group that were obtained by the emotion estimation unit 42, an emotion vector that is closest to the emotion vector of the receiver obtained by the emotion estimation unit 42, and selects a message that has the determined emotion vector. By obtaining an emotion vector for each message and the receiver information in this way, it is possible to compare the emotions of both the communicator and the receiver, and it is possible to facilitate the selection of a message.

Also, in the message selection device according to the first embodiment, for each message in the message group acquired by the message group acquisition unit 41, the emotion estimation unit 42 calculates a ratio of each emotion component included in an emotion indicated by the message, converts each emotion component into an emotion vector based on the calculated ratio of the emotion component, and acquires an emotion vector of the message by obtaining a sum of the emotion vectors of the emotion components. Accordingly, an emotion vector can be easily obtained for each message.

Note that in the message selection device according to the first embodiment, the emotion vectors can be a vector in Russel's circumplex model of affect in which emotions are mapped in a two-dimensional space defined by a valence axis and an arousal axis.

Also, the message presentation device according to the first embodiment includes the message selection device according to the first embodiment and the message presentation unit 50 that presents a message selected by the message selection unit 40 of the message selection device to a receiver. Accordingly, it is possible to present a message that is closer to the emotion of the receiver while also conveying the emotion of the communicator, and it is possible to express sympathy with the receiver in a short message.

### Second Embodiment

In the first embodiment, the message presentation device that includes the message selection device is configured as one device operated by the receiver. However, the message selection device or the message presentation device may be provided as a system divided into a plurality of devices.

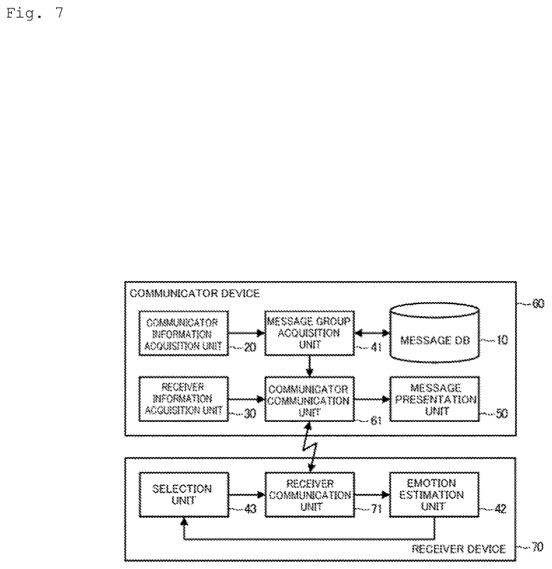

FIG. 7 is a block diagram showing an example of the configuration of a message presentation device that includes a message selection device according to a second embodiment of the present invention. As shown in FIG. 7, the message presentation device is constituted by two devices, namely a communicator device 60 in possession of the communicator and a receiver device 70 in possession of the receiver.

The communicator device 60 includes a message database 10, a communicator information acquisition unit 20, a receiver information acquisition unit 30, a message group acquisition unit 41 of a message selection unit 40, and a message presentation unit 50, which are similar to the corresponding units described in the first embodiment. The communicator device 60 further includes a communicator communication unit 61 that exchanges data with the receiver device 70. In the second embodiment, the communicator device 60 is envisioned to be a communication device for attachment to the collar of a pet such as a dog.

The receiver device 70 includes the emotion estimation unit 42 42 and the selection unit 43 of the message selection unit 40, which are similar to the corresponding units described in the first embodiment. The receiver device 70 further includes a receiver communication unit 71 that exchanges data with the communicator device 60. In the second embodiment, the receiver device 70 is envisioned to be a smartphone or a personal computer in possession of a person who is the owner of a pet such as a dog.

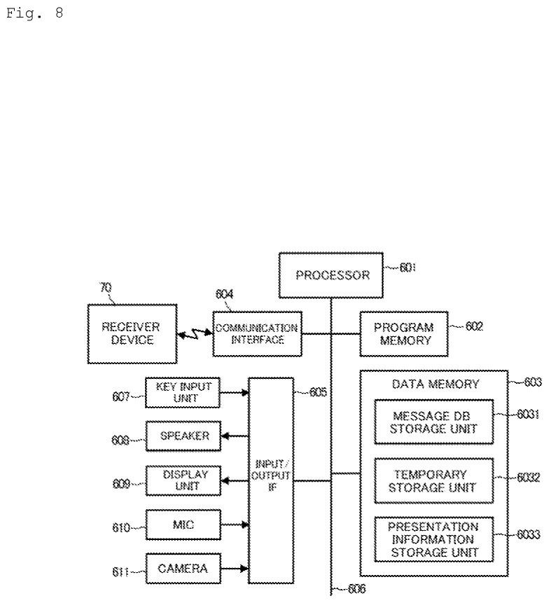

FIG. 8 is a diagram showing an example of the hardware configuration of an information processing device that constitutes the communicator device 60 in the message presentation device shown in FIG. 7. As shown in FIG. 8, the information processing device includes a hardware processor 601 as well as a program memory 602, a data memory 603, a communication interface 604, and an input/output interface 605 that are connected to the processor 601 via a bus 606. Note that in FIG. 8, “input/output interface” is abbreviated as “input/output IF”.

Here, the program memory 602 is a non-transitory tangible computer-readable storage medium that includes a non-volatile memory that can be written to and read from at any time, such as an HDD or an SSD, in combination with a non-volatile memory such as a ROM. The program memory 602 stores programs necessary for the processor 601 to execute various types of control processing pertaining to the second embodiment. Specifically, processing function units in the communicator information acquisition unit 20, the receiver information acquisition unit 30, the message group acquisition unit 41, the message presentation unit 50, and the communicator communication unit 61 can all be realized by the processor 601 reading out and executing a program stored in the program memory 602. Note that some or all of these processing function units may be realized in various other aspects, including an integrated circuit such as an ASIC, a DSP, or an FPGA.

Also, the data memory 603 is a tangible computer-readable storage medium that includes the above-mentioned non-volatile memory in combination with a volatile memory such as a RAM. The data memory 603 is used to store various types of data acquired and created during the execution of various types of processing. Specifically, areas for storing various types of data are appropriately secured in the data memory 603 during the execution of various types of processing. As examples of such areas, the data memory 603 may be provided with a message database storage unit 6031, a temporary storage unit 6032, and a presentation information storage unit 6033. Note that in FIG. 8, “message database storage unit” is abbreviated as “message DB storage unit”.

The message database storage unit 6031 stores a plurality of messages in correspondence with each of various communicator emotions. Specifically, the message database 10 can be configured in the message database storage unit 6031.

The temporary storage unit 6032 stores various types of data, such as data that is acquired or generated when the processor 601 performs operations as the communicator information acquisition unit 20, the receiver information acquisition unit 30, and the message group acquisition unit 41, as well as communicator information, receiver information, message groups, and emotions.

The presentation information storage unit 6033 stores messages that are to be presented to the receiver when the processor 601 performs operations as the message presentation unit 50.

As one example, the communication interface 604 includes a wireless communication module that utilizes short-range wireless technology such as Bluetooth. This wireless communication module performs wireless data communication with the receiver device 70 Under control of the processor 601. In other words, the processor 601 and the communication interface 604 can function as the communicator communication unit 61.

Also, a key input unit 607, a speaker 608, a display unit 609, a microphone 610, and a camera 611 are connected to the input/output interface 605. Note that in FIG. 8, “microphone” is abbreviated as “MIC”.

The key input unit 607 includes buttons and operation keys such as a power key for causing the communicator device 60 to start operating. The input/output interface 605 inputs operation signals to the processor 601 in accordance with operations performed on the key input unit 607.

The speaker 608 generates sound in accordance with a signal received from the input/output interface 605. For example, the processor 601 converts a message stored in the presentation information storage unit 6033 into audio information, and the audio information is input to the speaker 608 as an audio signal by the input/output interface 605, and thus the message is presented to the receiver as audio. In other words, the processor 601, the input/output interface 605, and the speaker 608 can function as the message presentation unit 50.

The display unit 609 is a display device that uses a liquid crystal display, an organic EL display, or the like, and displays images that correspond to signals received from the input/output interface 605. For example, the processor 601 converts the message stored in the presentation information storage unit 6033 into image information, and the image information is input to the display unit 609 as an image signal by the input/output interface 605, and thus the message can be presented to the receiver as an image. In other words, the processor 601, the input/output interface 605, and the display unit 609 can function as the message presentation unit 50.

The microphone 610 collects nearby sounds and inputs them as an audio signal to the input/output interface 605. Under control of the processor 601, the input/output interface 605 converts the received audio signal into vocalization information and stores it in the temporary storage unit 6032. The microphone 610 collects vocalizations emitted by the communicator and the receiver. Accordingly, the processor 601 and the input/output interface 605 can function as the communicator information acquisition unit 20 and the receiver information acquisition unit 30.

The camera 611 captures images in the field of view and inputs a captured image signal to the input/output interface 605. Under control of the processor 601, the input/output interface 605 converts the received captured image signal into image information and stores the image information in the temporary storage unit 6032. When the communicator device 60 is attached to the communicator, if the camera 611 is attached so as to captures images ahead of the communicator, the camera 611 can capture images of the receiver. Accordingly, the processor 601 and the input/output interface 605 can function as the receiver information acquisition unit 30 for acquiring receiver image information.

The input/output interface 605 may have a function for reading from and writing to a recording medium such as a semiconductor memory (e.g., a flash memory), or may have a function for connection to a reader/writer having a function for reading from and writing to such a recording medium. Accordingly, a recording medium that can be mounted to and removed from the information processing device can be used as the message database storage unit that stores messages. The input/output interface 605 may further have a function for connection with other devices such as a biosensor that detects biometric information of the communicator.

Also, the information processing device that constitutes the receiver device 70 may have the hardware configuration shown in FIG. 1B described in the first embodiment. However, the message database storage unit 1031 is not required in the data memory 103. The program memory 102 stores programs necessary for the processor 101 to execute various types of control processing pertaining to the second embodiment. Specifically, processing function units in the emotion estimation unit 42, the selection unit 43, and the receiver communication unit 71 can all be realized by the processor 101 reading out and executing a program stored in the program memory 102.

Next, operations of the message presentation device that includes the message selection device according to the present embodiment will be described.

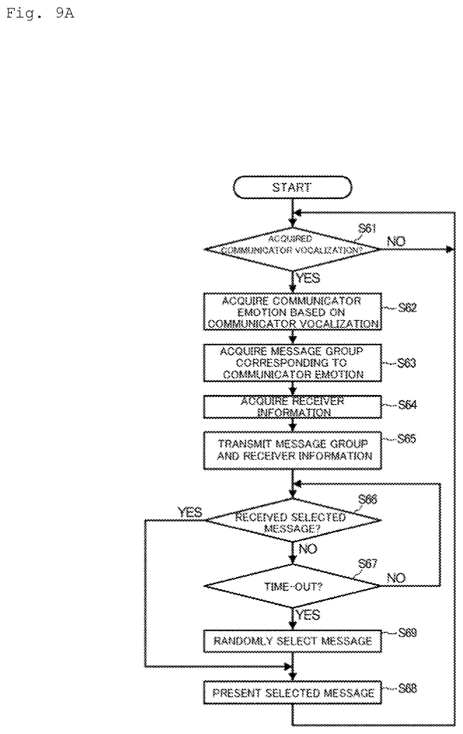

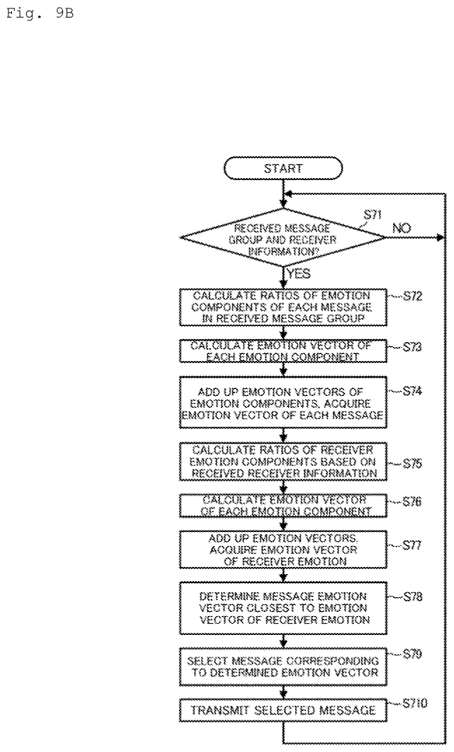

FIG. 9A is a flowchart showing an example of processing operations in the information processing device that constitutes the communicator device 60 in the message presentation device. This flowchart shows processing operations in the processor 601 of the information processing device that functions as the communicator information acquisition unit 20, the receiver information acquisition unit 30, the message group acquisition unit 41, the message presentation unit 50, and the communicator communication unit 61 of the communicator device 60. For example, if the communicator device 60 is attached to the communicator (e.g., is attached to a dog's collar or the like) and then an instruction to start message presentation is given via the input/output interface 605 due to an ON operation performed on the power key of the key input unit 607, the processor 601 starts the operations shown in this flowchart.

First, the processor 601 functions as the communicator information acquisition unit 20 and determines whether or not a communicator vocalization collected by the wireless microphone 610, that is to say dog's bark, has been acquired by the input/output interface 605 (step S61). Here, if it is determined that a communicator vocalization has not been acquired (NO in step S61), the processor 601 repeats the processing of step S61.

On the other hand, if it is determined that a communicator vocalization has been acquired (YES in step S61), the processor 601 stores the acquired communicator vocalization in the temporary storage unit 6032 and performs operations as the message group acquisition unit 41.

Specifically, first, the processor 601 acquires a communicator emotion, such as a dog emotion, based on the communicator vocalization stored in the temporary storage unit 6032 (step S62). There are no particular limitations on the method used to acquire the communicator emotion in this embodiment.

The processor 601 then acquires a message group that corresponds to the acquired communicator emotion from the message database 10 stored in the message database storage unit 6031 and stores the acquired message group in the temporary storage unit 6032 (step S63).

Next, the processor 601 functions as the receiver information acquisition unit 30 to acquire receiver information (step S64). For example, as the receiver information, a receiver vocalization collected by the microphone 610 and/or a face image of the human receiver captured by the camera 611 is stored in the temporary storage unit 6032 by the processor 601 via the input/output interface 605.

Subsequently, the processor 601 performs operations as the communicator communication unit 61.

Specifically, first, the processor 601 transmits the message group and the receiver information stored in the temporary storage unit 6032 to the receiver device 70 via the communication interface 604 (step S65).

The processor 601 then determines whether or not a selected message was received from the receiver device 70 by the communication interface 604 (step S66). Here, if it is determined that a selected message has not been received (NO in step S66), the processor 601 determines whether or not a time-out has occurred, that is to say whether or not a preset time has elapsed (step S67). If a time-out has not yet occurred (NO in step S67), the processor 601 repeats the processing from step S66. Note that the preset time is determined based on the time required for the processing of selecting a message in the receiver device 70.

FIG. 9B is a flowchart showing an example of processing operations in the information processing device that constitutes the receiver device 70 in the message presentation device. This flowchart shows processing operations in the processor 101 of the information processing device that functions as the emotion estimation unit 42, the selection unit 43, and the receiver communication unit 71 of the receiver device 70. For example, when the start of message presentation is instructed by the key input unit 107 via the input/output interface 105, the processor 101 reads out a program stored in the program memory 602 and starts the operations shown in this flowchart.

First, the processor 101 functions as the receiver communication unit 71 and determines whether or not a message group and receiver information have been received from the communicator device 60 via the communication interface 104 (step S71). Here, if it is determined that a message group and receiver information have not been received (NO in step S71), the processor 101 repeats the processing of step S71.

On the other hand, if it is determined that a message group and receiver information have been received (YES in step S71), the processor 101 stores the received message group and receiver information in the temporary storage unit 1032, and then performs operations as the emotion estimation unit 42.

Specifically, first, for each of the messages included in the message group stored in the temporary storage unit 1032, the processor 101 calculates the ratios of the emotion components indicated by the message (step S72). There are no particular limitations on the method for calculating the ratios of emotion components in this embodiment. Next, the processor 101 converts the calculated emotion components into an emotion vector for each message (step S73). The processor 101 then acquires an emotion vector for each message by obtaining the sum of the emotion vectors of the emotion components of the message (step S74).

The processor 101 then calculates the ratios of the emotion components of the human being who is the receiver based on vocalization information and/or a face image that constitutes the receiver information stored in the temporary storage unit 1032 (step S75). There are also no particular limitations on the method for calculating the ratios of emotion components of the receiver in this embodiment. Next, the processor 101 converts the calculated receiver emotion components into an emotion vector (step S76). The processor 101 then acquires an emotion vector of the receiver emotion by obtaining the sum of the emotion vectors of the emotion components (step S77).

In this way, after the emotion vector of each message of the message group and the emotion vector of the receiver emotion have been acquired, the processor 101 performs operations as the selection unit 43.

Specifically, first, the processor 101 determines the message emotion vector that is closest to the emotion vector of the receiver emotion (step S78). The processor 101 then selects the message that has the determined message emotion vector (step S79). The processor 101 stores the selected message in the presentation information storage unit 1033.

After a message has been selected from the message group in this way, the processor 101 functions again as the receiver communication unit 71 and transmits the message stored in the presentation information storage unit 1033 to the communicator device 60 as the selected message (step S710).

The processor 101 then repeats the processing from step S71.

The communicator device 60 receives the selected message from the receiver device 70 via the communication interface 604 and stores the generated message in the presentation information storage unit 6033. Accordingly, the processor 601 determines that a selected message has been received (YES in step S66). The processor 601 then functions as the message presentation unit 50 and presents the selected message stored in the presentation information storage unit 6033 by, via the input/output interface 605, outputting the message as audio with use of the speaker 608 or outputting the message as an image to the display unit 609.

Subsequently, the processor 601 repeats the processing from step S61.

On the other hand, if a time-out occurs before a selected message is received from the receiver device 70 (YES in step S67), the processor 601 randomly selects a message from the message group stored in the temporary storage unit 6032 (step S69). The processor 601 then stores the message that was selected in the presentation information storage unit 6033 as the selected message.

Subsequently, the processor 601 proceeds to the processing of step S68 and presents the selected message, which is the randomly selected message.

The message selection device according to the second embodiment described above includes: a communicator device 60 in possession of the communicator and a receiver device 70 in possession of the receiver, and the receiver device 70 includes at least the emotion estimation unit 42 and the selection unit 43 of the message selection unit 40. In this way, the portions that require high-performance and high-speed processing functionality are implemented in a smartphone or personal computer that includes a high-performance processor 101, and thus a low-functionality processor can be used as the processor 601 of the communicator device 60, and the communicator device 60 can be provided at low cost.

Also, if the communicator device 60 does not receive a selected message from the receiver device 70, the communicator device 60 presents one of the messages included in the selected message group, and thus a receiver who does not have the receiver device 70 can be presented with a message similar to that in conventional technology based on only the communicator emotion.

### Third Embodiment

In the first and second embodiments, the ratios of the emotion components of a message are calculated by the emotion estimation unit 42. However, the emotion components may be calculated in advance for each message registered in the message database 10.

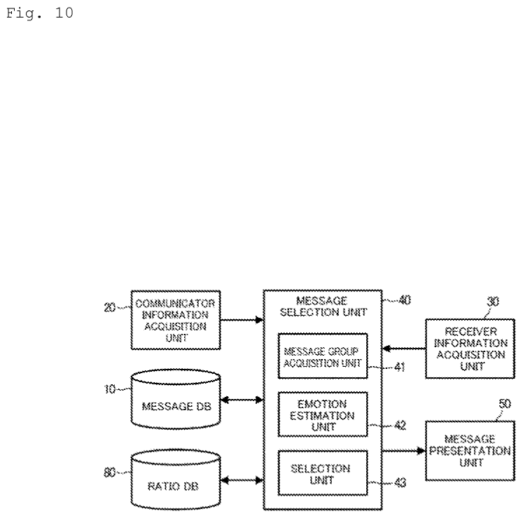

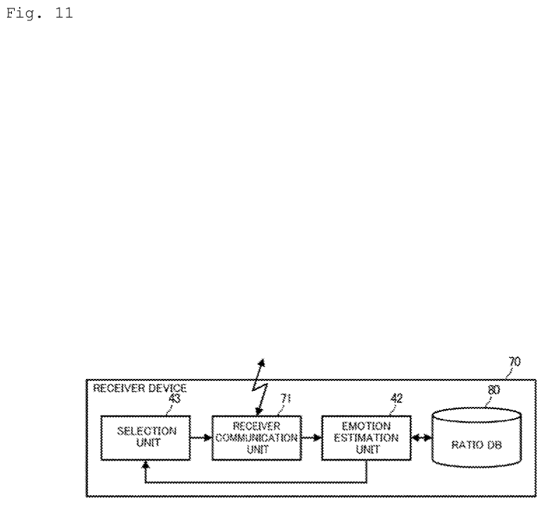

FIG. 10 is a block diagram showing an example of the configuration of a message presentation device that includes a message selection device according to a third embodiment of the present invention. In this configuration, a ratio database 80 has been added to the configuration of the message presentation device in the first embodiment. Also, FIG. 11 is a block diagram showing the configuration of the receiver device 70 in another example of the configuration of the message presentation device that includes the message selection device according to the third embodiment. In this configuration, the ratio database 80 has been added to the configuration of the receiver device 70 in the second embodiment. Note that in these figures, “ratio database” is abbreviated as “ratio DB”.

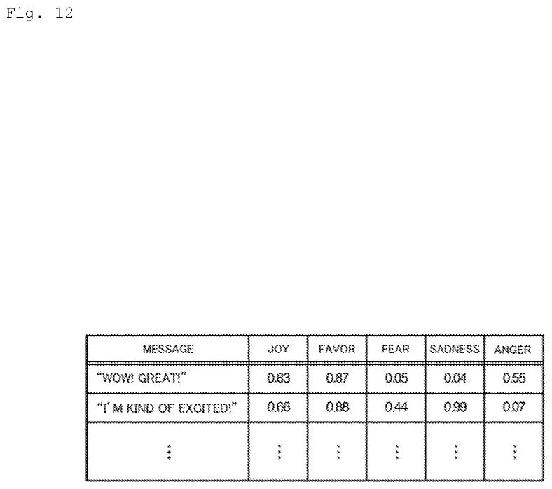

FIG. 12 is a diagram showing an example of information held in the ratio database 80. As shown in FIG. 12, the ratio database 80 holds emotion components that have been calculated in advance for each message. Accordingly, the emotion estimation unit 42 can acquire the emotion components of a message without calculating the emotion components.

The message selection device according to the third embodiment described above further includes: a ratio database 80 configured to hold, for each of the messages that correspond to the emotion of the communicator, a ratio of each emotion component indicated by the message, wherein for each message in the message group acquired by the message group acquisition unit 41, the emotion estimation unit 42 converts each emotion component indicated by the message into an emotion vector based on the ratios of the emotion components of the message held in the ratio database 80, and acquires an emotion vector of the message by obtaining a sum of the emotion vectors of the emotion components. Accordingly, it is not necessary to calculate emotion components for each of a plurality of messages, and therefore the processing speed can be increased.

### Other Embodiments

In the first to third embodiments described above, examples are described for the case of estimating the emotion of a human being who is the receiver based on vocalization information or a face image, but the present invention is not limited to this. For example, there are various proposals for techniques for estimating human emotions based on various types of information such as speech content from a receiver acquired by a microphone and biometric information such as a heart rate acquired by a biometric sensor, such as in JP 2014-18645A and JP 2016-106689A.

Also, in the operations described in the first to third embodiments, communication between a dog and a person is described as an example, but the present invention is not limited to this application. Various embodiments are also applicable to communication with a communicator who cannot express emotions as words, such as communication between a person and another type of pet such as a cat or a bird, and communication between a human infant and a parent.

Also, in the operations described in the first to third embodiments, emotion vectors are used to calculate the similarity between the emotions of the communicator and the receiver, but the similarity between the emotions of the communicator and the receiver may be calculated using another indicator.

Also, although the emotion vectors are defined in Russell's circumplex model of affect, the emotion vectors may be defined using another emotion model.

Also, the sequences of the processing steps shown in the flowcharts of FIGS. 3, 9A, and 9B are merely examples, and the present invention is not limited to these sequences. For example, in FIG. 3, step S7 for acquiring receiver information may be performed between steps S1 and S8. Also, if the processor 101 is capable of performing parallel processing, the processing of step S7 may be executed in parallel with the processing of steps S2 to S6. In this way, the time at which a processing step is performed may be changed as long as there is no conflict with preceding or subsequent processing steps.

Also, some of the functions of the information processing device that constitutes the message selection device or the message presentation device may be constituted by a server device on the network 400. For example, the message database 10 and the message selection unit 40 can be provided in the server device.

Also, all the functions of the message selection device or the message presentation device may be provided in the server device. In this case, if the function of collecting communicator information and receiver information and the function of outputting a selected message are provided as skills, a smart speaker connected to the network 400 can be presented to the receiver as if it were a message presentation device. For example, a smart speaker having only a microphone and a speaker as a user interface can transmit vocalization information from the communicator and the receiver to the server device via the network 400, receive a selected message from the server device via the network 400, and output corresponding audio using the speaker. As another example, a smart speaker having a camera and a display as a user interface can transmit vocalization information and face image information regarding a receiver to the server device via the network 400, receive a selected message from the server device via the network 400, and output corresponding audio using the speaker, or displaying the message using the display.

Also, the techniques described in the above embodiments can be realized by a program (software means) that can be executed by a computer, and can be stored on a recording medium such as a magnetic disk (floppy (registered trademark) disk, hard disk, etc.) an optical disk (CD-ROM, DVD, MO, etc.), or a semiconductor memory (ROM, RAM, flash memory, etc.), or be transmitted and distributed via a communication medium. Note that the program stored on a medium can also be a setting program for configuring, in a computer, the software means (including not only an execution program but also a table or a data structure) to be executed by the computer. A computer that realizes this device reads the program recorded on the recording medium, or constructs the software means using the setting program in some cases, and executes the above-described processing by performing operations under control of the software means. Note that the recording medium referred to in the present specification is not limited to being for distribution, and includes storage media such as a magnetic disk or a semiconductor memory provided in a computer or in a device connected via a network.

In other words, the present invention is not limited to the above embodiments, and can be modified in various ways at the implementation stage without departing from the gist of the invention. Also, the embodiments may be carried out in combination as appropriate, in which case a combined effect can be obtained. Moreover, inventions at various stages are encompassed in the above-described embodiments, and various inventions can be extracted from appropriate combinations of the disclosed constituent elements.

### REFERENCE SIGNS LIST

10 Message database (message DB)
20 Communicator information acquisition unit
30 Receiver information acquisition unit
40 Message selection unit
41 Message group acquisition unit
42 Emotion estimation unit
43 Selection unit
50 Message presentation unit
60 Communicator device
61 Communicator communication unit
70 Receiver device
71 Receiver communication unit
80 Ratio database (ratio DB)
101, 601 Processor
102, 602 Program memory
103 Data memory
1031, 6031 Message database storage unit (message DB storage unit)
1032, 6032 Temporary storage unit
1033, 6033 Presentation information storage unit
104, 604 Communication interface
105, 605 Input/output interface (input/output IF)
106, 606 Bus
107, 607 Key input unit
108, 608 Speaker
109, 609 Display unit
110, 610 Microphone (MIC)
111, 611 Camera
200 Wireless microphone (MIC)
300 Sensor group
400 Network (NW)

1. A message selection device comprising:
a message database configured to hold a plurality of messages in correspondence with an emotion of a communicator;
a processor; and
a storage medium having computer program instructions stored thereon, when executed by the processor, perform to:
acquire communicator information for estimating the emotion of the communicator;
acquire receiver information for estimating an emotion of a receiver who is to receive a message from the communicator; and
estimate the emotion of the communicator based on the communicator information, estimate the emotion of the receiver based on the receiver information, and select a message from the plurality of messages held by the message database based on the estimated emotions.

2. The message selection device according to claim 1, wherein the computer program instructions further perform to
acquire, from the message database, a message group that corresponds to the communicator information;
estimate an emotion indicated by each message in the message group, and estimate an emotion indicated by the receiver information; and
select a message that is closest to the emotion of the receiver, based on the emotions indicated by the messages in the message group.

3. The message selection device according to claim 2,
wherein the computer program instructions further perform to converts each message in the message group into an emotion vector, and converts the receiver information unit into an emotion vector, and
determines, from among the emotion vectors of the messages in the message group, an emotion vector that is closest to the emotion vector of the receiver, and selects a message that has the determined emotion vector.

4. The message selection device according to claim 3,
wherein the computer program instructions further perform to, for each message in the message group, calculates a ratio of each emotion component included in an emotion indicated by the message, converts each emotion component into an emotion vector based on the calculated ratio of the emotion component, and acquires an emotion vector of the message by obtaining a sum of the emotion vectors of the emotion components.

5. The message selection device according to claim 3, further comprising:
a ratio database configured to hold, for each of the messages that correspond to the emotion of the communicator, a ratio of each emotion component indicated by the message,
wherein the computer program instructions further perform to, for each message in the message group converts each emotion component indicated by the message into an emotion vector based on the ratios of the emotion components of the message held in the ratio database, and acquires an emotion vector of the message by obtaining a sum of the emotion vectors of the emotion components.

6. The message selection device according to claim 3,
wherein the emotion vectors are each a vector in Russel's circumplex model of affect in which emotions are mapped in a two-dimensional space defined by a valence axis and an arousal axis.

7. The message selection device according to claim 2,
wherein the message selection device includes a communicator device in possession of the communicator and a receiver device in possession of the receiver, and
the receiver device includes at least the emotion estimation unit and the selection unit of the message selection unit.

8. A message presentation device comprising:
the message selection device according to claim 1; and
a message presentation unit configured to present, to the receiver, the message selected by the message selection unit of the message selection device.

9. A message selection method in a message selection device that includes a processor and a message database holding a plurality of messages in correspondence with an emotion of a communicator, and selects a message that corresponds to the emotion of the communicator from the messages held in the message database, the message selection method comprising:
acquiring communicator information for estimating the emotion of the communicator, by the processor;
estimating the emotion of the communicator based on the acquired communicator information, by the processor;
acquiring receiver information for estimating an emotion of a receiver who is to receive a message from the communicator, by the processor;
estimating the emotion of the receiver based on the acquired receiver information, by the processor; and
selecting a message from the messages held in the message database, based on the estimated emotions of the communicator and the receiver, by the processor.

10. A non-transitory computer-readable medium having computer-executable instructions that, upon execution of the instructions by a processor of a computer, cause the computer to function as the message selection device according to claim 1.
